SEQUENCE LISTING (1 sequence)

SEQ ID NO: 1 - protein, 19 aa, synthetic construct
GERRQYFWIAWYKLANSKK

(19) United States
(12) Patent Application Publication
(10) Pub. No.: US 2024/0317806 A1
(43) Pub. Date: Sep. 26, 2024

(54) TOPICAL PEPTIDE FORMULATION FOR THE TREATMENT OF SEXUAL DYSFUNCTION

(71) Applicant: Biozeus Biopharmaceuticals S.A., Ipanema (BR)

(72) Inventors: Paulo Gustavo Sampaio Lacativa, Lagoa (BR); Gabriela Westerlund Peixoto Neves, Leblon (BR); Camilla Nunes dos Reis Trindade, Niteroi (BR); Joaquim Herzog de Oliveira Knox, Botafogo (BR)

(73) Assignee: Biozeus Biopharmaceuticals S.A., Ipanema (BR)

(21) Appl. No.: 18/443,717

(22) Filed: Feb. 16, 2024

Related U.S. Application Data
(60) Provisional application No. 63/485,866, filed on Feb. 17, 2023.

(51) Int. Cl. C07K 7/08; A61K 9/00; A61K 38/00; A61K 47/10; A61P 15/10

(52) U.S. Cl. CPC C07K 7/08; A61K 9/0014; A61K 47/10; A61P 15/10; A61K 38/00

(57) ABSTRACT

Aspects of the disclosure relate to topical pharmaceutical formulations, which comprise a Nitric Oxide Synthase-inducer (NOS) synthetic peptide and permeation-enhancing mixture of excipients. In some embodiments, the formulations provide enhanced peptide absorption and penetration through the skin and mucosa relative to previously described formulations. In some aspects, the disclosure provides a method of preparing such formulations. In some embodiments, the disclosure provides methods for treating sexual dysfunctions in humans using the formulations.

### RELATED APPLICATIONS

This application claims the benefit under 35 U.S.C. 119(e) of U.S. provisional Application Ser. No. 63/485,866, filed Feb. 17, 2023, entitled “TOPICAL PEPTIDE FORMULATION FOR THE TREATMENT OF SEXUAL DYSFUNCTION,” the entire contents of which are incorporated by reference herein.

### REFERENCE TO AN ELECTRONIC SEQUENCE LISTING

The contents of the electronic sequence listing (B168470000US01-SEQ-KZM.xml; Size: 1,759 bytes; and Date of Creation: Feb. 16, 2024) is herein incorporated by reference in its entirety.

### FIELD

The disclosure relates to topical pharmaceutical formulations and methods for treating sexual dysfunctions in a subject.

### BACKGROUND

Sexual dysfunction is a common condition in which man and women experience difficulties during any stage of normal sexual activity, which may result in serious impact on the individual's quality of life and may cause high levels of personal or interpersonal distress.

### SUMMARY

PnPP-19 is a Nitric Oxide synthase (NOS)-enhancer, increasing the levels of Nitric Oxide (NO), a potent vasodilator. PnPP-19 is an unstable peptide. Moreover, PnPP-19 needs to reach the tissue target (e.g., foreskin of penis, and smooth muscle layer of penis and vagina) to induce the vasodilatation of the corpus cavernosum (CC), and be useful for the treatment of sexual dysfunction. However, PnPP-19 can cause vasodilatation in non-desirable tissues if it reaches the systemic vessels.

Aspects of the disclosure relate to compositions and methods for treating sexual dysfunction. The disclosure is based, in part, on topical formulations comprising a therapeutic synthetic peptide, PnPP-19 as set forth in SEQ ID NO: 1, and other pharmaceutically acceptable excipients, such as a permeation-enhancing, stabilizer(s) and surfactant(s). It has been surprisingly discovered that formulations described by the disclosure are not only useful for the treatment of sexual dysfunction in a subject (e.g., male subjects and female subjects), but that such formulations have several improved properties, for example improved stability (e.g., shelf-life) and permeability of target tissue, fast onset of action, a pleasant sensation upon application, and reduced skin and/or mucosal irritation relative to previously described formulations. In some embodiments, these improved properties are important factors for increasing patient compliance when using the formulations described herein.

Unexpectedly, it has been found that therapeutic peptides derived from Phoneutria nigriventer toxin (e.g., PnPP-19) can be formulated with other excipients, such as propylene glycol and surfactants, in certain ratios to provide a substantially increased absorption and bioavailability of the peptides in the skin/mucosa of a subject.

The methods and formulations provided herein present several attractive features and desirable properties that make them suitable for use to treat a variety of conditions associated with sexual dysfunction, including pleasing sensations and ease of use.

Accordingly, in some aspects, the disclosure provides a topical pharmaceutical formulation comprising a synthetic peptide comprising or consisting of the amino acid sequence set forth in SEQ ID NO: 1 present in a concentration of between 0.1% and 0.7% (weight/volume) w/v; a surfactant; propylene glycol; and a buffer having a pH between 4.0 and 6.5.

In some aspects, the disclosure provides a topical pharmaceutical formulation comprising a synthetic peptide consisting of the amino acid sequence set forth in SEQ ID NO: 1 between 0.1 and 0.7% w/v; a surfactant in a concentration range of about between 2.5 and 18.0% w/v; propylene glycol in a concentration range of about between 7.5 and 30.0% w/v; and, a buffer in a concentration range of about between 50.0 and 73.5% (volume/volume) v/v, wherein the pH of the buffer is about between 4.0 and 6.5. In some embodiments, such formulations may be referred to herein as the “FAPP20”; “FAPP22.1”; FAPP22.2”; “FAPP22.3”; “FAPP22.4”; “FAPP22.5”; or “FAPP23.1” formulation.

In some embodiments, a surfactant comprises or consists of a poloxamer. In some embodiments, a poloxamer comprises Poloxamer 188, Poloxamer 407, or a combination thereof.

In some embodiments, a buffer comprises sodium acetate or potassium phosphate.

In some embodiments, a formulation further comprises one or more preservatives or antioxidants.

In some embodiments, a formulation further comprises one or more additional excipients. In some embodiments, one or more excipients comprise a filler, lubricant, wetting agent, flavoring agent, perfume, and/or emulsifier.

In some embodiments, a formulation is in the form of a cream, gel, lotion, ointment or spray.

In some embodiments, a formulation crosses the fibrous layer of a subject's tunica albuginea without exhibiting a systemic effect in the subject.

In some aspects, the disclosure provides a method of treatment of diseases that benefit from the modulation of the smooth muscle cells (SMC) contractility, comprising topically administering to a subject in need thereof, a formulation as described herein.

In some embodiments, the disclosure provides a method of treatment of male sexual dysfunction, comprising topically administering to a subject in need thereof, a formulation as described herein.

In some embodiments, male sexual dysfunction is selected from the group consisting of erectile dysfunction (ED), delayed ejaculation, premature ejaculation, and male hypoactive sexual desire disorder.

In some embodiments, the disclosure provides a method of treatment of female sexual dysfunction (FSD), comprising topically administering, to a subject in need thereof, a formulation as described herein.

In some embodiments, the FSD is selected from the group consisting of female sexual arousal disorder (FSAD), female orgasmic disorder, female hypoactive sexual desire disorder and genito-pelvic pain/penetration disorder. In some embodiments, the genito-pelvic pain/penetration disorder is dyspareunia or vaginismus.

In some embodiments, a formulation is administered to the skin, mucosa or urethra of the subject.

In some embodiments, a subject is male, and the formulation is administered to the penis or urethra of the subject.

In some embodiments, a subject is female, and the formulation is administered to the genitalia, such as the labia minora and majora, and clitoris of the subject.

In some embodiments, a formulation is applied daily. In some embodiments, a formulation is administered less than daily, for example once every 2, 3, 4, 5, 6, 7, or more days.

In some embodiments, a formulation is administered prior to (e.g., between 60 minutes and about 1 minute before) sexual activity.

In some aspects, the disclosure provides a topical pharmaceutical formulation as described herein for use in the treatment of diseases that benefit from the modulation of the SMC contractility.

In some aspects, the disclosure provides a topical pharmaceutical formulation as described herein for use in the treatment of sexual dysfunction in a subject.

In some embodiments, the sexual dysfunction is a male sexual dysfunction, selected from the group consisting of ED, delayed ejaculation, premature ejaculation and male hypoactive sexual desire disorder.

In some embodiments, the sexual dysfunction is a female sexual disorder (FSD), selected from the group consisting of female sexual arousal disorder (FSAD), female orgasmic disorder, female hypoactive sexual desire disorder and genito-pelvic pain/penetration disorder. In some embodiments, the genito-pelvic pain/penetration disorder is dyspareunia or vaginismus.

In some aspects, the disclosure provides a use of a pharmaceutical formulation as described herein for the preparation of a medicament for treating diseases that benefit from the modulation of the SMC contractility.

In some aspects, the disclosure provides a use of a pharmaceutical formulation as described herein for the preparation of a medicament for the treatment of sexual dysfunction in a subject.

In some embodiments, the sexual dysfunction is a male sexual dysfunction selected from the group consisting of ED, delayed ejaculation, premature ejaculation and male hypoactive sexual desire disorder.

In some embodiments, the female sexual dysfunction is a FSD selected from the group consisting of FSAD, female orgasmic disorder, female hypoactive sexual desire disorder and genito-pelvic pain/penetration disorder. In some embodiments, the genito-pelvic pain/penetration disorder is dyspareunia or vaginismus.

In some aspects, the disclosure provides a method of making a pharmaceutical formulation as described herein, the method comprising introducing to a pharmaceutically acceptable excipient a peptide consisting of the amino acid sequence set forth in SEQ ID No: 1, wherein the peptide is present in an amount between about 0.1% and 0.7% w/v. In some embodiments, pharmaceutical compositions as described herein have a fast onset of activity (e.g., within about between 5 and 30 minutes, for example within 15 minutes) after administration to the subject.

Additional embodiments and advantages of the disclosure will be set forth, in part, in the description that follows, and will flow from the description, or can be learned by practice of the disclosure. The embodiments and advantages of the disclosure will be performed and attained by means of the elements and combinations particularly pointed out in the appended claims.

### BRIEF DESCRIPTION OF DRAWINGS

FIG. 1 shows representative data indicating the release profile of the amount of PnPP-19 (%) in the FAPP20 formulation placed in the donor compartment.

FIG. 2 shows representative data indicating the permeation profile of PnPP-19 (%) in the FAPP20 formulation from the amount added in the donor medium.

FIG. 3 shows representative data for photodegradation of representative FAPP20 formulations with and without UV protection. Formulations were incubated for 96 hours in a UVA/UVB radiation chamber and the degradation of PnPP-19 was calculated based on a previous validated HPLC-DAD analytical method. (*) with UV protection.

FIG. 4 shows representative data for the kinetics of peptide PnPP-19 in target tissues. When applied in the FAPP20 formulation, the peptide is significantly detected in target tissues (e.g., foreskin and smooth muscle layer) in the first analysis period (15 minutes), demonstrating a fast release. In the smooth muscle layer, it remains stable for 30 minutes, while detection in the skin drops, and detection increases in the foreskin. Within 60 minutes, a small amount is detected in the tissues. The distribution of the peptide was determined by radioactivity in each tissue, relative to the administered dose, per gram of target tissue.

FIG. 5 shows representative data for the biodistribution of peptide PnPP-19, 15 minutes after application of FAPP20 formulation, demonstrating a fast release. Data shown are the result of normalization of % applied dose/gram tissue. For this calculation, the total value of the applied dose (sum of the quantification in all tissues analyzed) was considered 100% of the applied dose, and then the proportional value of the applied dose in each tissue was calculated. Thus, in the FAPP20 formulation, from 100% of the applied dose/gram in the animals' penis, approximately 42% is found in the skin, 18% is found in CC tissue (e.g., target tissue which comprises SMC) and 40% is found in the foreskin (e.g., target tissue which comprises dorsal nerves). The peptide was not detected in other organs or systemically.

FIG. 6 shows representative data indicating potentiation of erectile function by the PnPP-19 peptide administered topically in the FAPP20 formulation in healthy rats. Penile erection was induced by pelvic ganglion stimulation. ICP: intracavernous pressure. MAP: mean arterial pressure, ratio (ICP/MAP). N=8 per group. *=p<0.05, **=p<0.01, ***=p<0.001. (ANOVA One Way followed by Bonferroni).

### DETAILED DESCRIPTION

Aspects of the disclosure relate to compositions and methods for treating sexual function in a subject in need thereof. The disclosure is based, in part, on pharmaceutical compositions (also referred to herein as formulations) comprising peptides derived from the toxin PnTx2-6. In some embodiments, pharmaceutical compositions described by the disclosure are safe and effective in treating certain signs and symptoms (e.g., erectile dysfunction) associated with sexual dysfunction.

U.S. Pat. No. 9,279,004 (the entire contents of which are incorporated by reference herein) discloses a peptide with 19 amino acids (PnTx(19)) and molecular weight of 2,485.85 Da, built from the toxin PnTx2-6. The natural toxin, PnTx2-6, causes priapism in male patients bitten by the spider Phoneutria nigriventer. In turn, the peptide PnTx(19), also called PnPP-19, is a non-naturally occurring molecule, engineered from non-contiguous domains of the natural toxin PnTx2-6. In some embodiments, PnPP-19 is capable of enhancing the erectile function, as demonstrated by an improved relaxation of isolated strips of murine penile corpus cavernosum ex vivo. However, challenges exist for creating a formulation able to deliver the peptide PnPP-19 to target tissue in an effective amount; and at the same time without causing its systemic exposure.

The conventional route of therapy involving protein or peptide drugs, in general, is parenteral administration (i.e., by injection). This is primarily due to the lack of absorption of such drugs based on active peptides through the gastrointestinal tract. However, injections are painful and sometimes difficult to administer relative to other dosage forms. Moreover, parenterally injected peptides often have systemic exposure in subjects and therefore increase the risk of side effects. Oral delivery is preferable to injections for patient acceptance since it is less painful and more convenient for the patient. However, delivery of therapeutic (poly)peptides through the gastrointestinal tract has several problems such as low pH in the stomach, proteolytic degradation of peptide-based drugs in the small intestine, low absorption through the intestinal membrane, and limited stability of such formulations, especially as an aqueous solution, which are all potential barriers to absorption of (poly)peptides following oral administration. Moreover, oral delivery will not avoid the systemic exposure of the peptide.

Topical administration seems a good option to transdermally deliver peptide-based drugs through the skin or the mucosa. However, the formulations in which peptides reside suffer from a number of significant disadvantages that often preclude their practical application. Particularly, administration of formulations based on peptides can be quite challenging due to their lack of stability, degradation and loss of function of these molecules, in particular for long-term storage and transport.

Moreover, some auxiliary ingredients, like poloxamer, “trap” the peptide inside the formulation. This feature is important for parenteral delivery because it protects the peptide against thermal and enzymatic degradation, and slows the peptide release to the target tissue, improving its bioavailability. On the other hand, for transdermal delivery, this feature results in slow the onset of action and increases the peptide loss that stays in the skin trapped without permeation.

Another challenge with topical administration is the permeation through the skin or mucosa, in order to achieve delivery of the peptide to target tissue in an effective amount. Particularly in the case of transdermal delivery through the penis skin to reach the CC, the peptide needs to cross a very strong fibrous layer named the tunica albuginea, which is very difficult for peptides to permeate. Additionally, when peptide reaches well vascularized target tissue, it can become easily systemically available, a feature not desirable for medications intended to act only locally.

Finally, the topical formulations must have a good sensorial feeling for a subject and should not cause skin or mucosal irritation or sensitivity, in order to improve patient compliance.

All the foregoing characteristics are desired to be present at the same time in a topical formulation.

Aspects of the disclosure relate to peptide formulations (e.g., peptide formulations for treatment of sexual dysfunction in a subject) that may be delivered by a more desirable route (e.g., topical administration) to target tissue (e.g., reproductive organs, such as the penis or vagina), without causing unwanted systemic side effects. In some embodiments, compositions and methods described herein solve challenges associated with delivery of agents to treat sexual dysfunction, for example skin permeation, peptide “trapping”, and maintenance of stability and function of active ingredients (e.g., PnPP-19). In some embodiments, the compositions provide, for the first time, an active topical formulation, comprising the synthetic peptide PnPP-19, useful for the treatment of sexual dysfunction; which enhances the peptide stability; which does not “trap” the peptide, permitting it to have a fast onset of action; which has an optimal delivery—the peptide is able to reach the tissue target in an effective amount (e.g., overcoming the tunica albuginea), without systemic exposure; with good sensorial properties; and without causing skin/mucosal irritation or sensitivity.

Accordingly, in some aspects, the disclosure provides a topical pharmaceutical formulation comprising a synthetic peptide having the sequence set forth in SEQ ID NO: 1 (e.g., in a concentration range between 0.1 and 0.7% w/v); a surfactant (e.g., in a concentration range of about between 2.5 and 18.0% w/v); propylene glycol (e.g., in a concentration range of about between 7.5 and 30.0% w/v); and a buffer (e.g., in a concentration range of about between 50.0 and 73.5% v/v). In some embodiments, the buffer has a pH between about 4.0 and 6.5.

### Definitions

Unless otherwise explained, all technical and scientific terms used herein have the same meaning as commonly understood by one of ordinary skill in the art to which this disclosure belongs. Those skilled in the art are particularly directed to the reference “Current Protocols in Molecular Biology” for definitions and terms of the art (AUSUBEL, F. M., BRENT, R., KINGSTON, R. E., MOORE, D. D., SEIDMAN, G., SMITH, J. A., STRUHL, K. Current Protocols in Molecular Biology. John Wiley and Sons, Inc., Media Pa. 2015).

As used herein, the term “peptide” refers to a compound formed by a chain of amino acids that are linked by peptide bonds. The length of a peptide may vary. In some embodiments, a peptide is less than 50 amino acids in length. In some embodiments, a peptide ranges from about 5 to about 25 (e.g., 5, 6, 7, 8, 9, 10, 11, 12, 13, 14, 15, 16, 17, 18, 19, 20, 21, 22, 23, 24, or 25) amino acids in length. In some embodiments, a peptide is 5, 6, 7, 8, 9, 10, 11, 12, 13, 14, 15, 16, 17, 18, 19, 20, 21, 22, 23, 24, or 25 amino acids in length. Peptides may be naturally occurring peptides or peptides that are recombinantly or synthetically produced. In some embodiments, a peptide is recombinantly produced. The abbreviations for amino acid residues are the standard code of 3 letters and/or 1 letter used in the art for reference to one of the common 20-L amino acids. In some embodiments, a peptide comprises or consists of the amino acid sequence set forth in SEQ ID NO: 1.

The singular terms “a,” “an,” and “the” include plural referents unless context clearly indicates otherwise.

In the context of this specification, the term “comprising” means “including principally, but not necessarily solely”. Further, variations of the word “comprising” such as “comprise” and “comprises” have correspondingly varied meanings.

As used herein, the terms “treat”, “treating” and “treatment” refer to any indicia of success in the treatment or amelioration of an injury, disease, or condition, including any objective or subjective parameter such as abatement; remission; diminishing of symptoms or making the injury, disease, or condition more tolerable to the patient; slowing in the rate of degeneration or decline; or improving a patient's physical or mental well-being. The treatment or amelioration of symptoms can be based on objective or subject parameters, including the results of a physical examination, neuropsychiatric examinations, or psychiatric evaluation.

By an “effective amount” or a “therapeutically effective amount” of a drug or pharmacologically active agent is meant a nontoxic but sufficient amount of the drug or agent to provide the desired effect. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation. In some embodiments, an “effective amount” of a peptide (e.g., PnPP-19, SEQ ID NO: 1) ranges from about 0.5 mg/ml to about 8.0 mg/ml. In some embodiments, an effective amount of a peptide ranges from about 1 mg/ml to about 7 mg/ml. In some embodiments, an effective amount is about 1, 2, 3, 4, 5, 6 or 7 mg/ml. In some embodiments, an “effective amount” of a peptide (e.g., PnPP-19, SEQ ID NO: 1) in a formulation ranges from 0.1 and 0.7% w/v.

The term “excipient”, “additives” or “other ingredients or components” refers to a substance that is a non-therapeutic agent and is intended to be used as a carrier or medium for the delivery of a therapeutic agent or added to a pharmaceutical composition comprising the therapeutic agent. In some embodiments, an excipient enhances handling and storage properties, or allows or promotes the formation of a unit dose of a composition (e.g., a composition comprising a PnPP-19 peptide).

The term “topical administration” means administration of a substance for absorption through the skin, mucosa and tissues. Non-limiting examples of the formulation for topical administration include a cream, a gel, a lotion, emulsions, aqueous suspensions, an ointment, or a spray. In some embodiments, a formulation for topical administration is not a foam.

The term “sexual dysfunction” as used herein refers to any female or male sexual dysfunction including, without limitation, impairment in sexual desire, arousal, orgasm, or satisfaction, or premature ejaculation, any or all of which may be due to, for example, psychogenic, biologic (including vasogenic, endocrine related, menopause, and neurologic disorders), or medication-induced mechanisms, excluding pain or sexual paraphilias. The sexual dysfunction can be, for example, a disorder selected from the group consisting of a FSD, including FSAD, female orgasmic disorder, female hypoactive sexual desire disorder and genito-pelvic pain/penetration disorder (like dyspareunia and vaginismus); and male sexual dysfunction, including ED, delayed ejaculation, premature ejaculation, and male hypoactive sexual desire disorder.

As used herein, the term “about” means±10% of the specified value, unless otherwise indicated.

The term “at least” prior to a number or series of numbers is understood to include the number adjacent to the term “at least”, and all subsequent numbers or integers that could logically be included, as clear from context. When at least is present before a series of numbers or a range, it is understood that “at least” can modify each of the numbers in the series or range.

### Pharmaceutical Formulations

Aspects of the disclosure relate to pharmaceutical compositions comprising a PnPP-19 peptide (e.g., a peptide comprising or consisting of the amino acid sequence set forth in SEQ ID NO: 1). PnPP-19 is a synthetic, non-toxic peptide composed of 19 amino acid residues originating from the PnTx2-6 toxin, which was isolated from the venom of the Brazilian armadeira spider (Phoneutria nigriventer). In some embodiments, the isolated peptide (e.g., PnPP-19) is presented as a freeze-dried white powder (e.g., a solid physical form). In some embodiments, PnPP-19 is an amphipathic peptide with about 26% cationic amino acid residues. In some embodiments, PnPP-19 is positively charged at pH 7. In some embodiments, PnPP-19 peptide has a pI of 10.68 and pKa 12.37.

In some embodiments, pharmaceutical formulations of the disclosure comprise, in a pharmaceutically acceptable medium, an effective amount of a peptide having the amino acid sequence set forth in SEQ ID NO: 1. In some embodiments, the peptide is N-terminally acetylated, and/or C-terminally amidated. In some embodiments, the peptide is in the form of a pharmaceutically acceptable salt. Pharmaceutically acceptable salts of the peptide that may be used in the formulation of the compositions include, without limitation, acetate, chloride, bromide, carbonate, phosphate, palmitic acid, caproic acid, histidine, or trifluoroacetate. In some embodiments, the PnPP-19 peptide is acetylated, for example by incorporating an acetyl group into the N-terminus (e.g., peptide glycine (G)) of the peptide. In some embodiments, the PnPP-19 peptide is amidated, for example by incorporating an amide group into the C-terminus (e.g., peptide lysine (K)) of the peptide. In some embodiments, the PnPP-19 peptide is acetylated and amidated.

The peptides described herein can be prepared by any methodologies known by those skilled in the art, including recombinant and non-recombinant methods. Synthetic pathways (non-recombinant) include, without limitation, the chemical synthesis of the peptide in solid phase, the chemical synthesis of the peptide in liquid phase and biocatalyzed synthesis. In some embodiments, the peptides are obtained by chemical synthesis, in the liquid or solid phase, using manual, automated or semi-automated systems. Alternative embodiments and synthesis methods of peptides are described, for example in US Patent Application Publication US 2021/0060125, the entire contents of which are incorporated by reference herein.

The amount of the therapeutic peptide (e.g., PnPP-19 peptide, set forth as SEQ ID NO: 1) present in the formulation may vary. In some embodiments, the amount of peptide in a formulation ranges between about 0.5 mg/ml to about 8.0 mg/ml. In some embodiments, the amount of peptide in a formulation ranges from about 1 mg/ml to about 7 mg/ml. In some embodiments, the amount of peptide in a formulation is about 1, 2, 3, 4, 5, 6 or 7 mg/ml, depending on various factors such as the dosage of the peptide to be delivered, the indication to be treated, the individual patient, and the like. In some embodiments, the concentration of therapeutic peptide present in a formulation ranges from about 0.1 and 0.7% w/v (e.g., 0.1%, 0.2%, 0.3%, 0.4%, 0.5%, 0.6%, or 0.7% w/v). In some embodiments, the amount of PnPP-19 peptide will be a therapeutically effective amount, that is, an amount that will provide a therapeutic effect.

Aspects of the disclosure relate to pharmaceutical formulations comprising PnPP-19 peptide (e.g., as set forth in SEQ ID NO: 1) and a surfactant. The surfactant used in the formulation of the invention may be a conventional surfactant in the art. Exemplary surfactants that can be used with the present formulation are poloxamers or a mixture thereof. Topical formulations comprising poloxamers have been challenging to develop. For example, Akash et al. (2014) Polymer Reviews, 54:4, 573-597 disclose that topical administration of formulations comprising PF127 poloxamer resulted in incomplete protein release and skin irritation. In another example, International Application Publication No. WO2009/090558 discloses that topical foam compositions comprising poloxamers lack stability and collapse upon administration to a delivery site. The disclosure is based, in part, on the surprising discovery that a combination of PnPP-19 peptide and one or more poloxamers in a pharmaceutical formulation results in an increase in stability relative to previously described topical pharmaceutical formulations. In some embodiments, the surfactant comprises (or consists of) Poloxamer 188, or Poloxamer 407, or a mixture thereof. The concentration range of the surfactant present in the formulation may vary. In some embodiments, the concentration of surfactant (e.g., Poloxamer 188, Poloxamer 407, or a combination thereof) ranges from about 2.5% to about 18.0% as measured by weight/volume (w/v). In some embodiments, the concentration of Poloxamer 188 in a formulation (e.g., a formulation comprising a PnPP-19 peptide) ranges from about 2.5% to about 18.0% as measured by weight/volume (w/v). In some embodiments, the concentration of Poloxamer 407 in a formulation (e.g., a formulation comprising a PnPP-19 peptide) ranges from about 2.5% to about 18.0% as measured by weight/volume (w/v). In some embodiments, the total concentration of Poloxamer 188 and Poloxamer 407 in a formulation (e.g., a formulation comprising a PnPP-19 peptide) ranges from about 2.5% to about 18.0% as measured by weight/volume (w/v). In some embodiments, a pharmaceutical formulation comprises about 2.5%, 3%, 4%, 5%, 6%, 7%, 8%, 9%, 10%, 11%, 12%, 13%, 14%, 15%, 16%, 17%, or 18% surfactant (e.g., Poloxamer 188, Poloxamer 407, or a combination thereof), as measured by weight/volume (w/v).

In some embodiments, a pharmaceutical formulation comprises a buffer (e.g., an aqueous solution comprising one or more weak acids or bases, such as acetate or phosphate, and one or more cognate salts, such as sodium or potassium). In some embodiments, the buffer comprises sodium acetate and/or potassium phosphate. In some embodiments, the buffer comprises sodium acetate. In some embodiments, the buffer comprises potassium phosphate. The concentration range of the buffer in a formulation (e.g., a formulation comprising PnPP-19 peptide) may vary. In some embodiments, the concentration of buffer in the pharmaceutical formulation ranges between about 50.0% and about 73.5% as measured by volume/volume (v/v). In some embodiments, the concentration of buffer in the pharmaceutical formulation is about 50%, 55%, 60%, 65%, 70%, or about 75% as measured by volume/volume (v/v). In some embodiments, the concentration of buffer in the pharmaceutical formulation is about 60%, as measured by volume/volume (v/v).

Aspects of the disclosure relate to pharmaceutical formulations comprising PnPP-19 peptide (e.g., as set forth in SEQ ID NO: 1) and propylene glycol. Topical formulations comprising propylene glycol have been challenging to develop. For example, U.S. Pat. No. 6,019,988 discloses that formulations comprising propylene glycol and peptides resulted in poor stability for topical administration. The disclosure is based, in part, on the surprising discovery that a combination of PnPP-19 peptide and propylene glycol in a pharmaceutical formulation results in an increase in stability relative to previously described topical pharmaceutical formulations. In some embodiments, a pharmaceutical formulation comprises propylene glycol. In some embodiments, the concentration of propylene glycol in the formulation ranges from about 7.5% to about 30.0% as measured by weight/volume (w/v). In some embodiments, the concentration of propylene glycol in the formulation is about 7.5%, 8%, 10%, 15%, 20%, 25%, or 30% as measured by weight/volume (w/v).

In some embodiments, a pharmaceutical formulation has a pH ranging from between about 4.0 to about 6.5. In some embodiments, the pH of the pharmaceutical formulation is about 4.0, 4.5, 5.0, 5.5, 5.8, 6.0, 6.1, 6.2, 6.3, 6.4, or 6.5. In some embodiments, the pH of the pharmaceutical formulation is 5.0.

Pharmaceutical formulations of the disclosure may further include, in addition to the above pharmaceutical ingredients, additives or stabilizers for maintaining the stability of the peptide in the formulation and maintaining the function of the peptide. In some embodiments, the stabilizer is selected from one or more preservatives and one or more antioxidants. In some embodiments, the concentration of each stabilizer in the formulation is ranges from about 0.01 to about 2.0% m/m. Examples of preservatives include but are not limited to MICROCARE®PEHG e Euxyl®PE 9010 (phenoxyethanol and ethylhexylglycerin), MICROCARE®SB (Sodium Benzoate and Potassium Sorbate), SymOcide®PC (phenoxyethanol and caprylyl glycol), Nipaguard SCE (sorbitan caprylate, 1,3-propenodiol and benzoic acid). Examples of antioxidants include but are not limited to Vitamin E (tocopheryl acetate), Tinogard®TS (octadecyl di-t-butyl-4-hydroxyhydrocinnamate), Tinogard®TT (pentaerythrityl tetra-di-t-butyl hydroxyhydrocinnamate), BHT (butylated hydroxytoluene), and Ferulic Acid.

In some embodiments, a pharmaceutical formulation comprises one or more additional excipients, such as fillers, lubricants, wetting agents, flavoring agent, perfumes and emulsifiers. The amount of the additional excipients included in the pharmaceutical formulation of the invention is not particularly limited and may be appropriately adjusted within an amount range used in general formulation.

Pharmaceutical formulations of the disclosure can be prepared and formulated in accordance with the conventional methods such as disclosed, for example, in the British, European and United States Pharmacopocias (British pharmacopocia. Vol. 1. London: Medicines and Healthcare products Regulatory Agency; 2018; European pharmacopocia. 9th ed, Strassbourg: Council of Europe: 2018; United States Pharmacopocia, 42, National Formulary 37, 2018), Remington's Pharmaceutical Sciences (REMINGTON, J. P., AND GENNARO, A. R. Remington's Pharmaceutical Sciences. Mack Publishing Co., 18th ed. 1990), Martindale: The Extra Pharmacopoeia (MARTINDALE, W. AND REYNOLDS, J. E. F. Martindale: The Extra Pharmacopoeia. London, The Pharmaceutical Press 31st ed, 1996), Harry's Cosmeticology (HARRY, R., and ROSEN, M. R. Harry's cosmeticology. Leonard Hill Books, 9th ed. 2015), and in Prista's Pharmaceutical technology (PRISTA, L. V. N., ALVES, A. C., MORGADO, R. M. R. Técnica Farmacêutica e Farmácia Galênica. 4th ed. Fundação Calouste Gulbenkian. Serviço de Educação e Bolsas, 1996); each of the foregoing disclosures is incorporated by reference herein in its entirety.

In some embodiments, the formulations can be prepared by dissolving the peptide (e.g., PnPP-19 peptide), a solubility enhancer, and a buffer in water, adjusting the pH, if necessary, sterilizing by filtration, and aseptically filling into appropriate dispensers.

Pharmaceutical formulations of the disclosure may be in any pharmaceutically acceptable form for topical administration to the skin, mucosa or urethra, such as, for instance, a cream, a gel, a lotion, an ointment, or a spray, and the like. In some embodiments, the pharmaceutical formulation is a gel. Gels may provide a convenient and improved form for handling by the subject and allow for the preparation to contain a homogeneous mixture of ingredients.

In some embodiments, the pharmaceutical formulations described herein are formulated for immediate release of the active peptide. In some embodiments, a pharmaceutical formulation as described by the disclosure has a fast onset of activity, for example between about 5 minutes and 30 minutes (e.g., 5, 10, 15, 20, 25, or 30 minutes), after administration to a subject.

### Methods of Treatment

Aspects of the disclosure relate to methods of treating sexual dysfunction in a subject in need thereof. A subject may be a mammal. In some embodiments, the subject is a human, for example a male human or female human. In some embodiments, the methods comprise a step of topically administering a pharmaceutical formulation according to the disclosure to the subject. In some embodiments, the pharmaceutical formulation is self-administered by the subject.

Previous topical formulations for treatment of sexual dysfunction have faced challenges. For example, U.S. Pat. Nos. 6,284,735 and 5,576,290 disclose local (e.g., intravaginal, transmucosal, etc.) administration of melanocyte-stimulating hormone-like (MSH-like) peptides for treatment of sexual dysfunction. However, MSH-like peptides are understood to have systemic effects (e.g., act on neurons of the CNS of a subject) and are therefore less desirable than locally-acting therapeutic agents. The disclosure is based, in part, on the surprising discovery that topical formulations comprising PnPP-19 described herein act locally, with reduced systemic effects, relative to previously-described formulations for treatment of sexual dysfunction.

The amount of peptide (e.g., PnPP-19) in a pharmaceutical formulation administered to a subject may vary. In some embodiments, the amount of peptide in a formulation ranges between about 0.5 mg/ml to about 8.0 mg/ml. In some embodiments, the amount of peptide in a formulation ranges from about 1 mg/ml to about 7 mg/ml. In some embodiments, the amount of peptide in a formulation is about 2, 3, 4, 5, 6 or 7 mg/ml.

The disclosure is based, in part, on the use of pharmaceutical formulations described herein for the treatment of certain diseases that benefit from the modulation of the SMC contractility including, but not limited to, male and female sexual dysfunction.

In some aspects, sexual dysfunction is a disorder selected from, but not limited to, the group consisting of FSD, including FSAD, female orgasmic disorder, female hypoactive sexual desire disorder and genito-pelvic pain/penetration disorder (e.g., dyspareunia and vaginismus); and male sexual dysfunction, including ED, delayed ejaculation, premature ejaculation, and male hypoactive sexual desire disorder.

In some embodiments, pharmaceutical formulations described herein are applied topically and relax the SMC tissue of the genital area in humans. In some embodiments, administration of pharmaceutical formulations described herein results in relaxation of SMC tissue (e.g., SMC tissue of the genital area) of the subject.

In some embodiments, the pharmaceutical formulations of the disclosure are topically administered to the sexual mucosa, such as penis, vulva or other suitable portion of the skin, of the subject.

Surprisingly, it has been observed that pharmaceutical formulations disclosed herein, in some embodiments, promote the satisfactory delivery of peptides (e.g., PnPP-19 peptide) in a therapeutically effective amount to its action site in the SMC tissue of the penis. In some embodiments, administration of the pharmaceutical composition to a subject's penis results in the peptide crossing the tunica albuginea in the penis of the subject. In some embodiments, the formulation of peptides (e.g., PnPP-19) in pharmaceutical compositions described herein result in stabilization of the peptides, and a fast onset of action after administration to the subject (e.g., within between about 5 minutes and 30 minutes, for example within 15 minutes of administration to the subject).

In some embodiments, the frequency of administration to a subject varies, depending on the needs of each subject and the severity of the disease to be treated, with a recommendation of an administration both daily or whenever convenient for the subject. In some embodiments, a subject is administered a pharmaceutical formulation described herein once per day. In some embodiments, a subject is administered a pharmaceutical formulation as described herein prior to engaging in sexual activity (e.g., between 5 minutes before sexual activity and about 1 day before engaging in sexual activity.).

In some embodiments, a formulation is applied daily. In some embodiments, a formulation is administered less than daily, for example once every 2, 3, 4, 5, 6, 7, or more days.

In some embodiments, a formulation is administered prior to (e.g., between 60 minutes and about 1 minute before) sexual activity. In some embodiments, a formulation is administered about 60 minutes, 45 minutes, 30 minutes, 20 minutes, 15 minutes, 10 minutes, 5 minutes, or 1 minute before sexual activity.

### Method of Preparation

The disclosure also provides methods of making the pharmaceutical formulations described herein. In some embodiments, the methods comprise the steps of: introducing into a pharmaceutically acceptable excipient a peptide (e.g., a PnPP-19 peptide), wherein the peptide is present in an amount sufficient for the treatment of a sexual dysfunction in a subject. In some embodiments, the amount of peptide ranges from about 0.5 mg/ml to about 8 mg/ml (e.g., 0.5, 1, 2, 3, 4, 5, 6, 7, or 8 mg/ml).

Preferably, the method further comprises the step of processing the mixture into a formulation suitable for administration as topical application.

Preferably, the method of making the pharmaceutical formulation of invention provides a therapeutic formulation in the form a gel.

The disclosure is based, in part, on the surprising discovery that storage of formulations comprising PnPP-19 peptide in light-protective containers improves stability and preserves activity of the PnPP-19 peptide. In some embodiments, the disclosure relates to a container containing a pharmaceutical formulation (e.g., formulation comprising a PnPP-19 peptide). In some embodiments, the container is configured to block passage of light (e.g., UV light, visible light, infrared light, etc.) through the container. Examples of containers that block passage of light include but are not limited to metal (e.g., aluminum, etc.) or metal-lined containers, dark glass containers, and UV protective plastic containers. Any suitable container may be used, for example vials, jars, tubes, bottles, boxes, etc.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### EXAMPLES

The following examples are intended to provide those of ordinary skill in the art with a general description regarding how to implement and use the present invention and are not intended to be limitative of the scope of the invention in any manner, nor are they intended to mean that the experiments below are all the experiments implemented and the only experiments that can be implemented.

Sexual dysfunction has a multifactorial etiology, and its origin may stem from a variety of conditions, such as from biological, organic or psychological conditions, and/or social factors.

The prevalence often increases with age, and can affect, for instance, the penile erection, libido, desire, physical pleasure, sexual pain, arousal, and/or the ability to orgasm in humans.

In terms of the physiology, the penile erection and the clitoris engorgement, for instance, is a neurovascular event that depends on the integrity of the vascular, muscular and nervous substructures that constitute the penis and the clitoris. Sexual stimulation causes the release of neurotransmitters at the penile and vagina nerve endings that will cause increased blood flow resulting in erection and clitoris engorgement.

Nitric Oxide (NO) is the main neurotransmitter involved in penile erection and clitoris engorgement and is synthesized by the enzymes nitric oxide synthase (NOS). This enzyme has three main isoforms: the endothelial (eNOS), and the neuronal (nNOS) and inducible (iNOS). The eNOS and nNOS are called constitutive because they synthesize NO under physiological conditions. The iNOS is the inducible one, which is expressed only in the presence of a stimulus. All isoforms of this enzyme are detected in the penis, especially in the corpus cavernosum (CC). Their expression has been evidenced in the endothelium nerve terminals of the CC and neuronal bodies, mainly in parasympathetic, non-adrenergic and non-cholinergic (NANC) neurons and in several cellular structures.

The subsequent signaling cascade results in the relaxation of the cavernosal SMC, increase of the blood flow into those structures, and finally, the erection and clitoris engorgement. Failures in that mechanism may result in an impaired erection, insufficient for sexual intercourse, characterizing the ED; and the failure in female arousal, characterizing the FSAD.

There are two sets of nerves that innervate the penis and vagina: the cavernosum nerves, which are autonomic, and the dorsal nerves, which are somatic. The cavernosum nerves are predominantly autonomic and therefore controlled by the sympathetic/parasympathetic nervous system. When stimulated, they release acetylcholine and nNOS at their terminals. Acetylcholine stimulates the production of eNOS, which will produce NO. The nNOS directly produces NO at the nerve terminals.

The dorsal nerves, predominantly somatic, are more related to sensitivity, and are not able to cause erection per se, but dramatically enhance erection. These vessels have branches that are nNOS positive, meaning that their terminals will produce and release NO, aiding increased blood flow and consequently erection in man and clitoris engorgement in women.

Once produced, NO diffuses into the SMC adjacent to the CC, and initiates a chain reaction through binding and activation of soluble guanylate cyclase (GCs), which in turn catalyzes the breakdown of guanosine triphosphate (GTP) into cyclic guanosine monophosphate (cGMP). cGMP activates the enzyme cGMP kinase which will phosphorylate certain proteins and ion channels resulting in the opening and hyperpolarization of potassium channels, sequestration of intracellular calcium, inhibition of calcium channels, and blockage of calcium influx. With this, a drop in calcium levels occurs, resulting in SMC relaxation and vasodilation of arteries and arterioles, and consequently, increased blood flow to the region. This system is known as NO-GMPc.

The return to the flaccid state is initiated mainly by the hydrolysis of cGMP into guanosine monophosphate by phosphodiesterase type 5 (PDE5). In addition, detumescence also occurs due to the sympathetic discharge caused by ejaculation promoting vasoconstriction and inhibition of NO release by the CC and the endothelium. Thus, with the contraction of the arteries and the pressure on the veins decreased, the penis returns to its normal state.

The search for active drugs to treat sexual dysfunctions, such as ED, to enhance desire, sexual satisfaction, and improve the overall sexual life in a female or male subject has substantially increased over the years.

First-line therapy for ED is oral PDE5 inhibitors (PDE5i), such as sildenafil (Viagra®), vardenafil (Levitra®), tadalafil (Cialis®), avalafil (Spedra®). However up to 36% of patients with ED fail to respond to this treatment, do not tolerate PDE5i or have contraindications for its use.

Enhancement of the NO effect by inhibiting the PDE5-mediated hydrolysis of cGMP is the basis of this mechanism of action of this class of drugs. Therefore, PDE5i do not induce an erection. These drugs do not cause any effect on erectile function in the absence of sexual stimulation. PDE5i can potentiate an erection only if NO production is intact. Hence, PDE5i failure occurs especially due to impairment of NO production.

Endothelial dysfunction is the common denominator to many vascular risk factors that can lead to arteriogenic ED in man or FSAD in women due to impairment of eNOS production, resulting into a decrease of NO production. Atherosclerosis, hypertension (SAH), hyperlipidemia, cigarette smoking, diabetes mellitus (DM) and pelvic irradiation are some of the diseases related with endothelial dysfunction. Furthermore, patients that have undergone radical pelvic surgeries (e.g., radical prostatectomy (RP)) have an especially high risk of nerve injury and subsequent impairment of parasympathetic nervous system, resulting in a decrease of NO production.

With regard to the effect of RP on erectile function, it was formerly thought that post-RP penile mass loss was caused by hypoxia. The commonly seen arterial insufficiency, caused by intraoperative damage to the arteries supplying the CC, was thought to cause hypoxemia. This theory lost a lot of steam when it was discovered that the main damage was intracellular and it was not possible to prove a drop in oxygen levels to support this hypothesis, giving strength to the theory of neural injury.

RP can affect the cavernosum nerves, which pass posterolaterally to the prostate, either by direct injury (neurotomy), in surgeries in which the “nerve sparing” technique cannot be applied, or by neuropraxia, when the nerves are intact but “stunned” by surgical manipulation. The result in both cases is an acute loss of NO production.

Without NO production by the nerves (e.g., no nNOS action), and without stimulation for production by the vessels or by injury to them (e.g., no eNOS action), the only way to produce enough NO for an erection would be through iNOS. iNOS can produce significantly more NO than nNOS and eNOS, and moreover, inside the target cell itself. The problem is that this enzyme is only produced upon stimulus, such as injury or trauma. And in the case of RP, the penis is intact—the surgery takes place in the prostate, away from the CC.

At the cellular level, neural injury and the consequent lack of NO result in an increase in local cytokines and toxic agents that have a pro-apoptotic effect (cell death of SMCs) and a profibrotic effect (increased collagen production). The result is an atrophy of the cavernosum tissue, with loss of penile cell mass, and replacement by fibrous tissue. The loss of penile mass occurs to varying degrees and clinically will result in some loss of erectile function that may be partial or permanent. In this case, the injection of vasoactive substances within the CC of the penis fails to induce erection.

Some factors are related to a higher chance of the patient having sufficient erection for successful penetration in the future: patients younger than 60 years, good preoperative erectile function, and undergoing the “nerve sparing” technique have a higher chance of recovering erectile function. Other minor risk factors for not recovering erectile function are those related to atherosclerotic vascular disease: SAH, DM, hypercholesterolemia, and smoking.

Recovery, however, is slow. Those who recover have an improvement in erectile function for at least up to 24 months, and some cases as long as 48 months. In some instances, waiting 6 to 48 months for the period of recovery of erectile function is recommended.

The lack of NO production results in PDE5i failure in almost all individual submitted to RP, regarding the capacity to recover the erectile function.

In some embodiments, failure of PDE5i treatment encompasses inability to attain or maintain adequate penile erection during sexual intercourse on at least four consecutive occasions, despite optimum drug dosing. All PDE5i appear to have similar efficacy; therefore, if one drug treatment fails, then patients should be offered an alternative treatment based on a different class of compounds. Moreover, some patients may not be able to tolerate PDE5i because of adverse events related to vasodilatation in nonpenile tissue expressing PDE5, or from the inhibition of homologous nonpenile isozymes (i.e., phosphodiesterase type 6 in the retina). Finally, concomitant PDE5i use is contraindicated in nitrate users because it increases the risk of severe hypotension.

The combination of oral PDE5i with intracavernosum or intraurethral injection regimen has been observed to salvage as many as 31% of patients who do not respond to the therapies alone but has also been associated with an incidence of side effects in 33% of patients, including dizziness in 20% of them. Patients that do not respond to any medical treatment option might be candidate for penile implant surgery as third-line therapy.

Topical therapies, such as nitroglycerin, applied to the penis glans or penis shaft skin have been investigated, but none have demonstrated sufficient efficacy as required for FDA approval in ED, especially in those non-respondent to PDE5i. Accordingly, a safe, well-tolerated and efficacious treatment for ED, and which avoids the side effects discussed above, is desirable.

FSD is a complex multifaceted problem affecting at least one third of the female population. Estimates revealed that 40-43% of sexually active women have already reported sexual dysfunction, of which 33% in the age between 40 and 69 years-old.

FSAD, one type of FSD, is characterized by a recurrent inability to attain or maintain sufficient sexual arousal despite adequate stimulation. The main problem is a reduced NO production, resulting in an inability to increase blood flow following genital sexual arousal. The consequence is a lack of swelling of the labia and vaginal wall and a lack of clitoris enlargement and protrusion. There is no FDA-approved treatment for FSAD so far.

The following non-limiting examples describe pharmaceutical compositions and formulations that have been found to be safe and effective in treating sexual dysfunction.

### Example 1: Peptide Synthesis

The NO-inducer peptide (e.g., PnPP-19) of the present disclosure was chemically synthesized using Solid Phase Peptide Synthesis (SPFS) technique. The SPFS technique involves the use of a solid matrix, in which the peptide chain is constructed. In this process amino acids with protecting groups are used to prevent secondary reactions, such as the 9-fluorenylmethyloxycarbonyl group-Fmoc. In the end, the peptide is cleaved from the solid matrix and the protecting groups are removed, allowing the peptide of interest to be obtained. Thus, for the synthesis of PnPP-19, the solid polymeric matrix used was Rink amide resin (0.68 mmol/g), serving as a support for the coupling of the amino acid derivatives with the Fmoc group to obtain and protect the peptide sequence of PnPP-19. Furthermore, the peptide was N-terminally acetylated and C-terminally amidated.

### Example 2: Representative Formulations of FAPP20

Based on FAPP20 constitution, some variation in the excipients can be implemented in order to achieve other preferential formulations.

These modifications can be applied in order to vary sensorial parameters. In the table 1 below is described the constitution of FAPP20 formulation and the variations performed to achieve other preferential formulations. In all of these exemplary formulations the concentration of PnPP-19 varies from 1 mg/mL to 5 mg/mL.

All these formulations presented a peptide content in the specification range of 80.0 to 120.0% after 3 months of stability at room temperature (25° C.) (Table 1).

TABLE 1
Representative formulations

| | | | | Propylene | Poloxamer | Poloxamer | |
|---|---|---|---|---|---|---|---|
| Formulation | | Buffer | % Buffer | glycol | 407 | 188 | Preservatives |
| code | pH | Type | % (v/v) | % (w/v) | % (w/v) | % (w/v) | % (w/w) |
| FAPP20 | 5.0 | Sodium | 60 | 30 | 10 | 0 | 0 |
| | | Acetate | | | | | |
| FAPP22.1 | 5.0 | Sodium | 59.2 | 30 | 10 | 0 | 0.8 |
| | | Acetate | | | | | |
| FAPP22.2 | 5.8 | Potassium | 67.2 | 15 | 17 | 0 | 0.8 |
| | | Phosphate | | | | | |
| FAPP22.3 | 5.8 | Potassium | 64.7 | 15 | 17 | 2.5 | 0.8 |
| | | Phosphate | | | | | |
| FAPP22.4 | 5.8 | Potassium | 68.2 | 15 | 16 | 0 | 0.8 |
| | | Phosphate | | | | | |
| FAPP22.5 | 5.0 | Sodium | 68.2 | 15 | 16 | 0 | 0.8 |
| | | Acetate | | | | | |
| FAPP23.1 | 5.8 | Potassium | 60 | 30 | 10 | 0 | 0 |
| | | Phosphate | | | | | |

### Example 3: Preparation of FAPP20 Formulation

A gel formulation, in accordance with the present invention, was prepared by mixing the components, as shown in the Table 2 below:

TABLE 2
Examples of the composition of representative formulations

| | | Proportional |
|---|---|---|
| | | volume in the |
| Components | Concentration | formulation (%) |

(a) FAPP20
Part I - Sodium Acetate Buffer + PnPP-19

| | | | |
|---|---|---|---|
| 20 mM sodium acetate | 1.64 | mg/mL | 60% |

| | |
|---|---|
| buffer pH 5.8 | |
| PnPP-19 | 5 mg/mL (0.5% w/v) |

Part II - Addition of excipients + water (q.s.p.)

| | | | |
|---|---|---|---|
| Poloxamer 407 | 100 | mg/mL | 40% |
| Propylene glycol | 300 | mg/mL | |

(b) FAPP22.3
Part I - Potassium Phosphate Buffer + PnPP-19

| | | |
|---|---|---|
| 100 mM potassium | K₂HPO₄ - 1.48 mg/mL | 64.7% |
| phosphate buffer | KH₂PO₄ - 12.45 mg/mL | |
| pH 5.8 | | |
| PnPP-19 | 5 mg/mL (0.5% w/v) | |

Part II - Addition of excipients

| | | | |
|---|---|---|---|
| Poloxamer 407 | 170 | mg/mL | 34.5% |
| Poloxamer 188 | 25 | mg/mL | |
| Propylene glycol | 150 | mg/mL | |

Part III - Addition of preservative + water (q.s.p.)

| | | | |
|---|---|---|---|
| Microcare ® PEHG | 8 | mg/g | 0.8% |

(c) FAPP23.1
Part I - Potassium Phosphate Buffer + PnPP-19

| | | |
|---|---|---|
| 100 mM potassium | K₂HPO₄ - 1.48 mg/mL | 60% |
| phosphate buffer | KH₂PO₄ - 12.45 mg/mL | |
| pH 5.8 | | |
| PnPP-19 | 5 mg/mL (0.5% w/v) | |

Part II - Addition of excipients + water (q.s.p.)

| | | | |
|---|---|---|---|
| Poloxamer 407 | 100 | mg/mL | 40% |
| Propylene glycol | 300 | mg/mL | |

PEHG—Phenoxyethanol and Ethylhexylglycerin

The above-cited formulations are prepared as follow:

### Part I

### 20 mM Sodium Acetate Buffer pH 5.0+Peptide (PnPP-19)

Calculate the volume of sodium acetate with PnPP-19 solution necessary to obtain 60% of the final volume of the formulation. Weigh the sodium acetate to obtain a mass sufficient for a final solution of 20 mM. Then, measure and adjust the pH to 5.0 using NaOH or HCl. After this procedure, use the solution of sodium acetate to solubilize the peptide to achieve a concentration of 0.5% (w/v).

### OR

### 100 mM Potassium Phosphate Buffer pH 5.8+Peptide (PnPP-19)

Calculate the volume of potassium phosphate buffer with PnPP-19 solution necessary to obtain the targeted proportion of the final volume of the formulation. Weigh the K₂HPO₄ (solution 1) and KH₂PO₄ (solution 2) to obtain a mass sufficient for 1 M of each solution. Solution 1 and 2 have to be mixture in a proportion of 8.5% of solution 1 to 91.5% of solution 2, using ultrapure water to achieve a final concentration of 0.1 M of potassium phosphate buffer. Then, measure and adjust the pH to 5.8 using solution 1 or 2 to increase or decrease the pH, respectively.

After this procedure, use the potassium phosphate buffer to solubilize the peptide to achieve a concentration of 0.5% (w/v).

### Part II

### Propylene Glycol

Weigh the propylene glycol to obtain the targeted concentration (w/v) considering the final volume of the formulation. Mixture the propylene glycol with the solution containing sodium acetate and peptide until complete solubilization.

Poloxamer 407 and/or Poloxamer 188

Weigh the Poloxamer 407 and/or Poloxamer 188 to obtain the targeted concentration (w/v) considering the final volume of the formulation. Mixture the poloxamer to the solution containing sodium acetate, peptide and propylene glycol until complete solubilization. Adjust the final volume of the formulation, if necessary, with distilled water.

### Part III

### Microcare® PEHG

In case of addition of preservative in the formulation, this should be placed at the end of the process, in a final concentration of 0.8% (w/w).

### Solubilization of the Solution

The final solution should be stirred until complete solubilization. If necessary, use alternate methodologies to aid solubilization (agitation and ultrasonic bath—10 to 15 min). Afterwards, the formulation should be placed in the refrigerator for about 24 hours (without agitation). After this period, store the formulation at room temperature.

### Example 3: Determination of pH Range

The goal of this experiment was to evaluate the stability of the peptide across a wide range of PH values, in order to identify a favorable pH range.

Seventeen formulations were created with pH values ranged from 4.0 to 8.0. The PnPP-19 initial concentration in all preparations was 5 mg/mL. All of the preparations were filled into autoclaved glass vials and stopped using autoclaved stoppers. The vials were then sealed and placed onto stability of 5° C., 25° C. and 40° C. for 2 and 4 weeks. The preparations were tested with visual assessment (observation of the vial on the bench in ambient light), RP-HPLC, asymmetrical field-flow fractionation (AF4-MALLS), and osmolality.

According to table 3, the formulations with pH above 6.5 presented instable chemical profile, defined as a reduction in the peptide contend by more than 20% analyzed by RP-HPLC:

TABLE 3
Analysis of peptide contend in the preparations with different
pH, measured by RP-HPLC by comparison with the original
gradient, after 4 weeks stability at 25° C.

| Formulation number | pH | Peptide content |
|---|---|---|
| F01 | 4.6 | 96.4% |
| F03 | 5.2 | 96.7% |
| F07 | 5.6 | 96.3% |
| F12 | 6.4 | 96.0% |
| F13 | 7.0 | 76.8% |
| F14 | 7.5 | 66.1% |

In conclusion, in order to maintain purity and stability, the range of pH in the PnPP-19 formulations should be between 4 and 6.5.

### Example 4: Determination of the Buffer Species

The goal of this experiment was to evaluate the stability of the peptide across a wide range of buffer.

The presence of a buffer was essential to stabilize the peptide, as preparations without buffer became cloudy, with particles and increased viscosity.

After the introduction of the buffer, it is necessary to adjust the pH. When the acetic acid is used in order to adjust the pH, the formulations developed an unpleasant odor, inappropriate sensorial characteristic that jeopardize patient compliance. When NaOH or HCl is used to adjust the pH, the odor is neutral, and therefore NaOH and HCl are the substances selected for the formulation pH adjustment after the buffer introductions.

Seventeen formulations were created with different buffer species, including sodium acetate, tri sodium citrate dihydrate (citrate), L histidine, potassium phosphate and tris hydrochloride. The PnPP-19 initial concentration in all preparations was 5 mg/mL. All of the preparations were filled into autoclaved glass vials and stopped using autoclaved stoppers. The vials were then sealed and placed onto stability of 5° C., 25° C. and 40° C. for 2 and 4 weeks. The preparations were tested with visual assessment (observation of the vial on the bench in ambient light), RP-HPLC, AF4-MALLS, and osmolality.

According to table 4, citrate and Tris do not stabilize PnPP-19, presenting a reduction in the peptide contend by more than 10% analyzed by RP-HPLC. There is almost complete loss of peptide in F04 with citrate formulation.

TABLE 4
Analysis of peptide contend in the preparations with different
buffers, measured by RP-HPLC by comparison with the original
gradient, after 4 weeks stability at 25° C.,

| Formulation | | Visual | |
|---|---|---|---|
| number | Buffer | Assessment | Peptide content |
| F02 | Sodium Acetate | Clear | 96.4% |
| F04 | Citrate | Milky white | not possible |
| | | | to be measure |
| F09 | L Histidine | Clear | 96.7% |
| F12 | Potassium Phosphate | Clear | 96.0% |
| F16 | Tris Hydrochloride | Slight turbid | 76.7% |

Other preparations with different pH demonstrated that sodium acetate and potassium phosphate increase stability independent of pH; on the other hand, the effect of L-histidine was small compared to the pH effect.

In conclusion, in order to maintain purity and stability, the selected buffers to stabilize PnPP-19 were sodium acetate and potassium phosphate, and the necessary adjustment of pH after the introduction of the buffer must be performed with NaOH and HCl.

### Example 5: Determination of Additional Excipients

The goal of this experiment was to evaluate the stability of the peptide across a wide range of additional excipients.

Seventeen formulations were created with different additional excipients, including hydroxypropyl beta cyclodextrin (HPCD), alpha cyclodextrin, polyethylene glycol (PEG) 400, PEG 6000, poloxamer 407, mono ethyl ether (transcutol P), hydroxy ethyl Cellulose (HEC), polysorbate 20 (PS20) and microemulsion.

The PnPP-19 initial concentration in all preparations was 5 mg/mL. All of the preparations were filled into autoclaved glass vials and stopped using autoclaved stoppers. The vials were then sealed and placed onto stability of 5° C., 25° C. and 40° C. for 2 and 4 weeks. The preparations were tested with visual assessment (observation of the vial on the bench in ambient light), RP-HPLC, AF4-MALLS, and osmolality.

According to table 5, only alpha cyclodextrin and poloxamer 407 do not increase chemical degradation of PnPP-19 (a reduction in the peptide contend lower than 10% analyzed by RP-HPLC). Analyzes by SE-HPLC also confirmed that formulations with HEC, PEG 6000, PEG 400 and PS20 decrease physical stability. The microemulsion formulation was not possible to be analyzed by RP-HPLC due to technical limitations.

TABLE 5
Analysis of peptide contend in the preparations with different
additional excipients, measured by RP-HPLC by comparison with
the original gradient, after 4 weeks stability at 25° C.,

| Formulation number | Additional excipient | Peptide content |
|---|---|---|
| F06 | alpha cyclodextrin | 96.0% |
| F10 | HEC | 72.2% |
| F12 | Poloxamer 407 | 95.0% |
| F13 | PEG 6000, PS20 | 79.2% |
| F14 | PEG 6000, PEG 400 | 81.1% |
| F16 | microemulsion | not evaluated |

### Example 6: In Vivo Release and Permeation Study with FAPP20 Formulation

All of the additional excipients selected in the example 5, although have the effect to increase the peptide stability, can “trap” the peptide and avoid its release, preventing its permeation thought the skin.

The release and permeation experiments were carried out in order to determine whether PnPP-19 in the FAPP20 formulation, in microemulsion, formulated with alpha cyclodextrin or with Transcutol P was able to cross different physical barriers in a suitable quantity.

For the release test, a cellulose acetate membrane was used to generate the physical barrier between a donor compartment, where the formulations are added, and the recipient compartment, where the peptide is measured.

In the first permeation assay, the physical barrier was the whole pig skin, which mimics the human stratum corneum. In the second permeation test, the physical barrier was the Strat-M membrane, which mimics the human stratum corneum.

The assays were conducted in Franz vertical diffusion cells with 1.767 cm² of area for diffusion of the peptide, containing a donor compartment with a capacity of 1 mL and a recipient compartment with a capacity of 7 mL. To the recipient compartment, 7 mL of the biorelevant solution were added, in which the media selected by preliminary studies was 20 mM sodium acetate buffer pH 5.0+0.5% Tween® 20. To the donor compartment, 300 μL of the FAPP20, microemulsion, alpha cyclodextrin, and Transcutol P formulation were added. The cells were placed in a thermostatic bath, with a temperature of 37° C.±0.5° C., with the goal to mimic the surface temperature. The bioreceptor medium was subjected to 350 rpm agitation. In times 0.5-, 1-, 2-, 4-, 6- and 24 h, collections of 1 mL were made from the recipient compartment, in order to measure the peptide content.

For the release test, 3 controls were used to validate the assay, and they demonstrated that the PnPP-19 can be quantified in the receptor media, after 6 hours (peak) and until 24 hours, when the peptide was applied in the donor compartment, in saline or in the buffer of the different formulations.

In the first permeation assay, PnPP-19 was formulated with 6 μM of alpha cyclodextrin or with transcutol 15%. However, Pnpp-19 was not detected in the receptor media in those formulations, demonstrating that both of them “trap” the peptide, avoiding its release. In the second permeation assay, PnPP-19 was formulated in the FAPP20 or in microemulsion. 0.5% (w/v) PnPP-19 in microemulsion or in FAPP20 formulation was added in the donor compartment.

The release profile of PnPP-19 in the microemulsion showed a maximum value around 11%, which decreased abruptly after 4 hours of assay reaching 2% after 24 h.

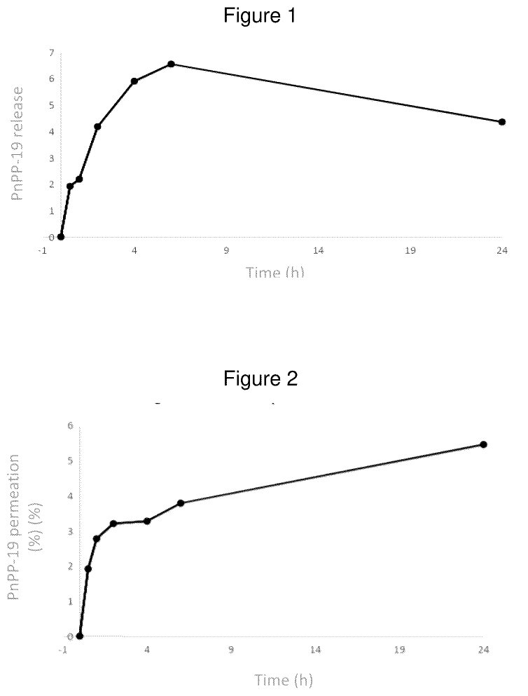

The release profile of PnPP-19 in the FAPP20 formulation showed a maximum value of 12% after 6 hours of assay, decreasing slowly and reaching 6% after 24 hours (FIG. 1).

Based on the release results described above, the 20 mM sodium acetate buffer pH 5.0+0.5% Tween® 20 was confirmed as the best receiving medium to be used in the permeation assay with Strat M membrane.

The permeation test was performed with one control that demonstrated the permeation of PnPP-19, when applied in the FAPP20 buffer (20 mM sodium acetate buffer solution), was detected in the receptor compartment since initial time (0.5 h).

0.5% w/v PnPP-19 in the microemulsion or in the FAPP20 formulation was added in the donor compartment:

PnPP-19 in the microemulsion formulation was not detected in the receptor media.

PnPP-19 in the FAPP20 formulation was detected since the initial time (0.5 h), in crescent concentrations until 24 hours, as shown in FIG. 2.

In conclusion, the FAPP20 formulation was able to allow a sustained release and permeation of PnPP-19 through the physical barriers for 24 hours. PnPP-19 formulated in alpha cyclodextrin, transcutol P or microemulsion “trap” the peptide, avoiding its release and permeation.

### Example 7: Long-Term Stability Analysis of Representative Formulations

The stability analysis of the PnPP-19 in the formulation FAPP20 and the other representative formulations was performed using a long-term protocol (temperature: 30° C.±2° C. and relative humidity (RH) of 75%±5%), in three different time-points: 0 (T0), 1 month (T1) and 3 months (T3). The stability protocols were performed according to Resolution 318/2019 from the Brazilian's regulatory agency (ANVISA—Agência Nacional de Vigilância Sanitária). For the content analysis, a validated high performance liquid chromatography (HPLC) method was used. Besides, the following criteria were evaluated: physical characteristics; pH; viscosity; weight loss; and microbiological analysis. The results are described in Table 6 below.

TABLE 6
Long-term stability analysis (30° C. ± 2° C. and RH 75% + 5%)
of the PnPP-19 in the FAPP20 formulation.

| Analysis | Method | Specification | Result T0 | | Result T1 | Result T3 |
|---|---|---|---|---|---|---|
| Characteristics | Visual | Clear transparent | In | | In | In |
| | inspection | gel | accordance | | accordance | accordance |
| pH | pH metro | Informative | 5.8 | | 5.7 | 5.7 |
| | reading | | | | | |
| Viscosity | Brookfield | Informative | 51.2 | cps | 43.0 cps | 45.9 |
| | Viscosimeter | | | | | |
| Content | HPLC | 80.0 to 120.0% | 95.4% | | 98.6% | 85.8% |
| Weight Loss | Weighing | Maximum 5% | NA | | 0.71% | 0.46% |
| | | No more than | | | | |
| | | 2.5% per year | | | | |
| | | (w/w) | | | | |
| Total count of | Colony count | Max 10² UFC/g | <10 | UFC/g | NA | <10 UFC/g |
| aerobic | (surface | | | | | |
| bacteria | analysis) | | | | | |
| Total fungal | Colony count | Max 10¹ UFC/g | <10 | UFC/g | NA | <10 UFC/g |

| | | | | | |
|---|---|---|---|---|---|
| count | (surface | | | | |
| | analysis) | | | | |
| Pathogen | Colony count | Absent | Absent | NA | Absent |
| research - | (surface | | | | |
| Staphylococcus | analysis) | | | | |
| aureus and | | | | | |
| Pseudomonas | | | | | |
| aeruginosa | | | | | |

Based on the results shown above, the PnPP-19 peptide remains stable (>80% of peptide content) after 3 months in the long-term stability condition.

Regarding the other listed representative formulations (Table 2), a long-term stability study was performed in the temperature of 25° C. for until 3 months. Each formulation was prepared three or six times (n=3-6). The results are presented at Table 7.

TABLE 7
PnPP-19 Long-term stability in representative formulations
at 25° C. for until 6 months. Results showed above indicate
the peptide content based on a validated HPLC-DAD method.

| ID | Specification | T0 | 1 month | 3 months |
|---|---|---|---|---|
| FAPP22.1 | 80.0 to 120.0% | 101.5% | 93.7% | 84.6% |
| FAPP22.2 | 80.0 to 120.0% | 97.9% | 99.6% | 98.9% |
| FAPP22.3 | 80.0 to 120.0% | 97.3% | 85.6% | 80.5% |
| FAPP22.4 | 80.0 to 120.0% | 99.7% | 99.9% | 90.8% |
| FAPP22.5 | 80.0 to 120.0% | 97.75% | 93.46% | 81.62% |
| FAPP23.1 | 80.0 to 120.0% | 99.84% | 98.21% | 90.03 |

### Example 8: Photodegradation of PnPP-19 in the Representative Formulations and Long-Term Stability with Light Protection

In order to evaluate the influence of UVA and UVB radiation on peptide degradation in the representative formulations, a photodegrading study was performed. For this assay the following formulations were tested: FAPP22.1 and FAPP22.4.

Therefore, each formulation was evaluated in two ways: I) With UV protection; II) Without UV protection. All samples were incubated in a UVA/UVB chamber (UVA source (95%)+UVB (5%)) for 96 hours. After this period, the samples were analyzed in HPLC-DAD using the same previously validated analytical methodology.

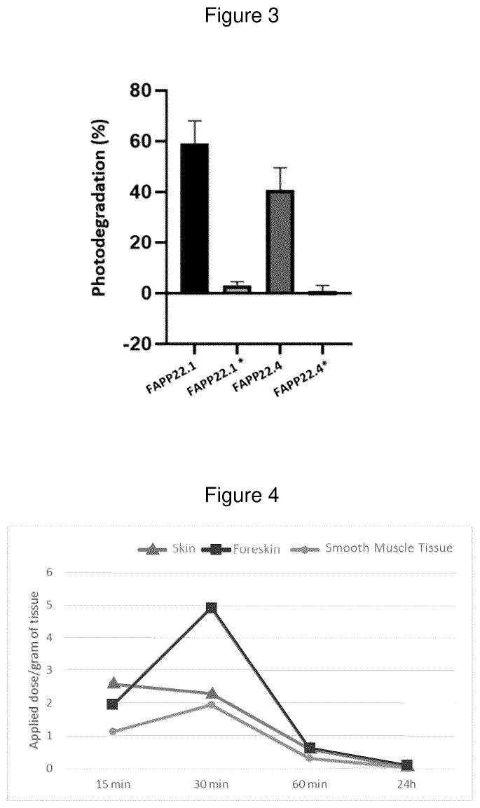

The results are shown in the FIG. 3 and table 8:

TABLE 8
Peptide degradation results for formulations FAPP22.1 and
FAPP22.4 after 96 h of incubation in a UVA/UVB radiation
chamber. Results are expressed as mean ± standard deviation.

| ID | With protection | Without protection |
|---|---|---|
| FAPP22.1 | 59.06% ± 8.99 | 3.11% ± 1.46 |
| FAPP22.4 | 40.81% ± 8.71 | 0.72% ± 2.3 |

In addition, a long-term stability study was performed with the formulation FAPP20 with and without light protection for 6 months in order to obtain the peptide content. As shown in Table 9:

TABLE 9
Long-term stability results of FAPP20 with and without
light protection after 6 months using HPLC validated
method according RDC no 166 (ANVISA).

| ID | With protection | Without protection |
|---|---|---|
| FAPP20 | 84.96% | 65.40% |

In conclusion, the formulation should be light protected in order to increase PnPP-19 stability.

### Example 9: Biodistribution and Pharmacokinetics after Topical Administration of PnPP-19, in FAPP20 Formulation, in Rats

In order to evaluate the in vivo permeation of the peptide (and/or its fragments) in the formulation FAPP20, a biodistribution study was carried out in healthy rats. The peptide was radiolabeled with iodine [¹³¹I] NaI using chloramine T method.

The study was conducted considering the dose of 5 mg/ml; and the maximum area of genital application. The final application volume was 100 μL that results in a dose of 2 mg/kg. The evaluation of biodistribution was carried out after the administration of a single dose of the peptide, applied topically to the skin of the penis after trichotomy of the rat's genital area.

Regarding biodistribution data, radiolabeled peptide concentrations were higher in skin, foreskin and SMC tissue at all times studied. In the first analyzed time, of 15 minutes, it was already possible to detect the peptide in the target tissues: the SMC layer, and the first layer of the skin, where the penile nerves are found (herein called of foreskin), showing that the peptide is rapidly absorbed into the skin. Within 30 minutes, its detection falls at the initial application site (skin), and increases in the foreskin, showing that it continues to be absorbed, and remains stable in the SMC layer. Within 60 minutes it is practically undetectable in tissues (FIG. 4).

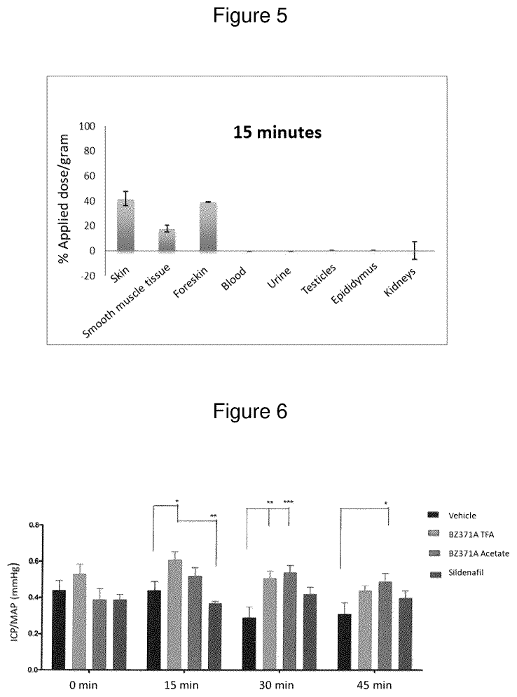

For the pharmacokinetic (PK) study, the blood of the animals was collected at 0 h (predose), 15, 30 and 60 minutes, 3, 8 and 24 hours. All samples collected were quantified in a Wizard 3 gamma counter (PerkinElmer) for later calculation of the percentage of dose applied per gram of organ. FIG. 5 shows another analysis of the biodistribution of the peptide PnPP-19 at 15 minutes time. While 42% of the applied dose is in the first layer of the skin, the peptide is detected in up to 18% in the SMC layer. Both peptide and its fragments were not found systemically (<1%) in the organs of choice (kidneys, testicles and epididymis) or in the blood, at any of the evaluated time-points, showing that the formulation remains in the application site, with no systemic absorption.

These data confirm that the FAPP20 formulation allows the peptide to rapidly permeate the skin, cross the tunica albuginea, and quickly reaching the target tissues in a significant amount. In addition, this biodistribution study confirmed the lack of systemic exposure of PnPP-19 or its fragments.

### Example 10: Effect of the Peptide PnPP-19, in the FAPP20 Formulation, on the Erectile Function of Healthy Rats

For this study, PnPP-19 was produced with TFA or Acetate as a counter-ion. Usually, peptides with TFA as a counter-ion are used in the preclinical stage of development; nevertheless, for safety purposes, the counter-ion is changed to acetate for clinical stage of development. Therefore, this experiment was performed to evaluate the efficacy of PnPP-19 in the FAPP20 formulation on the erectile function of healthy rats. Also, to compare if the counter-ion exchange would impact the biological effect of PnPP-19, two peptide batches was produced: one with the counter-ion TFA and another with the counter-ion Acetate. Sildenafil was used as a positive control (subcutaneously administration).

Erectile function was evaluated by means of the intracavernosal pressure/mean arterial pressure (ICP/MAP) ratio at 4 different times (0, 15, 30 and 45 minutes) after application of ganglionic electrical functional stimulus (EFS) of 8 Hz. Healthy adult male Sprague Dawley rats were separated into 4 groups, each with 8 animals: (i) negative control, treated with the proposed formulation (FAPP20) without the peptide (vehicle) and with saline applied subcutaneously (SC); (ii) peptide PnPP-19 with acetate counter-ion, formulated in FAPP20; (iii) peptide PnPP-19 with TFA counter-ion formulated in FAPP20; (iv) positive control, sildenafil administered via SC.

The formulated peptide (both with TFA counter-ion and Acetate) was applied topically to the penis of the rats 15 minutes before ICP/MAP measurements, at a dose of 1000 μg diluted in 200 μL of the formulation (dose of 4 mg/Kg), reaching a concentration of 5 mg/mL or 0.5% w/v of the peptide in the formulation. Sildenafil was applied SC, at a dose of 25 mg/Kg (1 mL/Kg) 30 minutes before ICP/MAP measurements.

Sildenafil was not superior to the negative control group (vehicle), although it showed a trend at the 30-minute time point, which was not statistically significant. Both peptide with TFA and peptide with acetate, in the FAPP20 formulation, showed superiority over the control, especially at the 15- and 30-minute time-points, and there was no statistical difference between them (FIG. 6). The conclusion is that the peptide, with both counter-ions, in the FAPP20 formulation is effective in potentiating erection, with a fast onset of action.

Again, no sign of dermal irritation was observed, and there was no difference in MAP between the groups treated with the peptide and vehicle, confirming that the peptide does not cause systemic hypotension in rats when applied topically. On the opposite, the sildenafil-treated groups had a significant decrease in MAP when compared to the vehicle-treated group.

In conclusion, these data demonstrated that PnPP-19, in the FAPP20 formulation, has a fast onset of action, reaches the target tissue in an effective amount, does not cause systemic hypotension and does not have local side effects (no sign of dermal irritation).

TABLE 10
MAP of rats after peptide PnPP-19 or sildenafil
administration compared to the vehicle.

| Group | 0 min | 15 min | 30 min | 45 min |
|---|---|---|---|---|
| Vehicle | 104 ± 10 | 106 ± 14 | 117 ± 13 | 117 ± 11 |
| PnPP-19-TFA | 105 ± 20 | 109 ± 16 | 117 ± 18 | 117 ± 20 |
| formulated in | | | | |
| FAPP20 | | | | |
| PnPP-19-Acetate | 94 ± 13 | 106 ± 18 | 108 ± 20 | 108 ± 20 |
| formulated in | | | | |
| FAPP20 | | | | |
| Sildenafil | 87 ± 10 (**) | 93 ± 8 (*) | 93 ± 8 (***) | 94 ± 7 (***) |

Results in mmHg, presented as the mean ± standard deviation.
(*) = p < 0.05;
(**) = p < 0.005;
(***) = p < 0.0008, compared to vehicle (Unpaired T-test)

### Example 11: Sub-chronic Toxicity and Toxicokinetic after the Daily Administration of PnPP-19, in FAPP20 formulation, for 60 Days in Pigs

A sub-chronic toxicity and toxicokinetic study was performed after administration of the peptide in the formulation FAPP20 in Brazilian Minipigs (male and female). This study was conducted in GLP, at the facilities of the Centro de Estudos Pré-Clinicos of the Instituto Israelita de Ensino e Pesquisa Hospital Albert Einstein.

This species was chosen based on the literature that describes minipigs as the best model for preclinical studies with topical drugs. The skin of this animal is classified as the most similar to human skin, both with respect to structure and pharmacokinetics (Bode, 2010).

The topical route was selected because it was the intended route in the clinical studies. The site of application, the genital region (penis or vulva and entrance to the vaginal canal) was selected for the same reason. Regarding the dose, as the investigational product in question does not have a defined maximum tolerable dose (MTD) or maximum no observed adverse effect dose (NOAEL), it was decided that this study would be conducted considering the maximum feasible dose (MFD) of the peptide in the FAPP20 formulation (0.7% w/v or 7 mg/mL) and the maximum application volume (the largest possible area of application in the genital region of pigs, standardized at 1 mL). Thus, the dose used was 7 mg per pig, which is equivalent to 0.7 mg/Kg.

The study duration of 60 days was selected to support the phase 2 clinical trial that will have the same duration. The number of pigs per group was selected according to the OECD 409 guide. A recovery group was included to determine, if a side effect had been detected, its resolution and reversibility. Thus, the experimental groups were: (1) peptide in the FAPP20 formulation (daily application of 7 mg of the peptide in 1 mL of for 60 days); (2) peptide in the FAPP20 formulation-Recovery (daily application of 7 mg of peptide in 1 mL of the formulation for 60 days, with an additional 7 days of recovery without drug application), and (3) Vehicle (daily application of formulation FAPP20 without the peptide for 60 days).

Each pig was observed at least once a day for clinical signs (including skin/hair; respiratory, urinary, and nervous system; feeding; locomotion, defecation, salivation, emesis, and others), morbidity, and mortality. The evaluation of water and feed consumption was qualitative. Body weight was monitored weekly. Blood samples were collected weekly (under general anesthesia) for evaluation of toxic effects over time (moments 1-10), with a baseline sample collected for animal control (moment 1), and included analysis of hematology, biochemistry, coagulogram, and urinalysis. In addition, blood samples were collected after the peptide application for toxicokinetic analysis on day 1 and day 60, and the detection and quantification of the peptide and its fragments were investigated. For this analysis, blood was collected at the following time intervals on day 1 and 60:0 (pre-dose), 15 min, 30 min, 60 min, 3 h, 8 h, and 24 h.

At the end of the study, the pigs were euthanized at the times defined for each group, through anesthetic overdose. During necropsy, macroscopic examination of all organs was performed and for histopathological examination the following organs were collected: liver with gallbladder, kidneys, adrenals, testes, epididymites, ovaries, uterus, thyroid (with parathyroid), spleen, brain and heart.

No mortality or morbidity was observed in the male and female groups during the entire period of the experiment.

As for clinical changes, as the episodes in males were rare and isolated, with spontaneous resolution, occurring in both groups randomly and at different weeks of treatment, they were considered as not clinically significant.

In females, diarrheal episodes occurred only in the treated group. The pasty diarrhea started from the 3rd week of treatment and continued until the 7th week of treatment. During the recovery period there were also no episodes of mushy defecation. Therefore, the mushy defecation was considered clinically relevant, but reversible and self-resolving.

As for the hematology, coagulogram and biochemistry, both in males and females, there were only isolated and punctual alterations, considered not clinically significant.

As for histology, no clinically significative difference was observed between the treatment groups and the vehicle group, both in the female and male pigs. One male in the treated group had suppurative posthitis; one female in the treated group had vascular lesion on mucosal surface; and one female in the vehicle group had inflammation of apocrine glands. All of these histological findings had no clinical or laboratory correlation and because they were isolated, they were considered not clinically significant.

Finally, regarding the toxicokinetic analysis, the peptide and its fragments were not detected in the blood of the pigs at any of the analyzed times.

Thus, it was concluded that the peptide PnPP-19 in FAPP20 formulation of the invention did not present toxic effects or dermal local side effects in pigs. The only adverse event considered relevant was the occurrence of pasty defecation in females, with more episodes in the treated group, but reversible and of spontaneous resolution. According to the toxicokinetic analysis, the product tested did not show systemic exposure in both sexes (males and females).

### Example 12: Texture Analysis of Representative Formulations

The sensorial of the representative formulations of the invention was tested with a texture assay.

The texture assays were performed using CT310K Texture Analyzer (Brookfield, EUA) in compression mode using the flat base plate probe TA-DEC. This probe is usually used to determine firmness values of materials. In this assay, the probe was forced down into each sample at a defined rate (2 mm/s) and to a defined depth (1 mm). When a trigger force of 2 g has been achieved, the probe penetrates the sample to a depth of 1 mm. During this time, the force to penetrate the sample increased. When the specified penetration distance was reached, the probe was withdrawn from the sample at the post-test speed of 2 mm/s. The maximum force value of the graph is a measure of the firmness of the sample at the specified depth. The minimum force value of the graph on the probe's return is a measure of adhesiveness.

The firmness parameter can generate insight into the spreading behavior of each formula since a formulation with high firmness has more resistance to spreading and vice-versa. Firmness (or hardness) is the force required until the material presents the required deformation. Adhesiveness is the work required to overcome the attractive forces of surfaces between the sample and the probe.

The test was performed in quadruplicate at a temperature range of 28.2° C.±0.4 above the gellification temperature of samples.

The FAPP22.1 formulation showed texture closer to water (Table 11), demonstrating that the FAPP22.1 formula is fluid. The FAPP22.4 formulation had an increase in firmness, around 600 g, and it also had higher adhesiveness, around 13 mJ. Both are adequate for topical application, if it is desirable to be less or more resistant to spreading on the skin and/or mucosa. These observations corroborate the results obtained from rheologic studies. The behavior of the FAPP22.4 formulation on the skin will follow that of a semi-solid gel that will remain intact when applied to the skin. By adding spreading forces, the initial tension of the material is broken, and it begins to flow, followed by a reduction in viscosity and, consequently, becomes more spreadable. In the other hand, the material of FAPP22.1 formulation will begin to flow over the skin immediately after application.

TABLE 11
Firmness and adhesiveness values of FAPP22.1 and FAPP22.4
compared to water (28.2° C. ± 0.4).

| | Firmness (g) | Adhesiveness (mJ) |
|---|---|---|
| Water | 23.00 ± 0.00 | 0.30 ± 0.00 |
| FAPP22.1 | 34.50 ± 1.76 | 0.43 ± 0.14 |
| FAPP22.4 | 604.20 ± 87.70 | 13.28 ± 1.50 |

Additionally, both FAPP22.1 and FAPP22.4 are translucent, without any undesirable smell, with homogenous appearance. Those characteristics are important to improve compliance.

### Example 13: Safety and Pharmacokinetics of FAPP20 Formulation in Human Trials

A pharmaceutical formulation as described by the disclosure was administered to human subjects during a Phase I trial, named: “Phase 1 clinical study to evaluate safety and pharmacokinetics of BZ371A in the form of gel applied to the genitalia of healthy men and women.” The objective of the study was to determine the safety and tolerability of BZ371A when applied topically to the genitalia of men and women, and to determine the systemic presence of BZ371A and its fragments through blood evaluation for pharmacokinetics, when BZ371A is topically applied in the genitalia of men and women.

BZ371A was administered in the formulation FAPP20, at a concentration of 5 mg/mL, topically applied to the genitalia (in men: whole penis, including glans; in women: clitoris, majora and minora labia), in a volume of 1.5 mL.

The study consisted of 3 visits and duration of 7 days. Medical and laboratory analyses were performed for safety assessment; and blood collections were performed for pharmacokinetic analysis. Quantification and detection of BZ371A and its fragments in the blood were obtained by a proper analytical method, specially developed for this purpose, and previously validated.

Twelve healthy adult subjects were included in the study (6 men and 6 women), without restriction regarding ethnicity, class, or social group, and were all allocated in a group. Only subjects in which the FAPP20 was properly applied were included in the analysis.

For the safety and tolerability analysis of BZ371A, the following outcomes will be considered: (1) adverse events report (AE); (2) local physical examination in the genital region; (3) vital signs, including systemic blood pressure and heart rate; electrocardiogram; and laboratory examination, including complete blood count (including platelets), coagulogram, biochemical (including glucose, sodium, potassium, urea, creatinine, chlorine, total calcium, phosphorus, total proteins and fraction (albumin and globulin), asparte transaminase, alanine transaminase, alkaline phosphatase, gamma-glutamyl transferase, total cholesterol, HDL cholesterol, triglycerides, pH; and urinalysis.

Related to the pharmacokinetic analysis, the following outcomes were considered: detection of BZ371A, and its fragments, in the blood at the following collected times: pre-dose, 15, 30, 60, 180 and 360 minutes; if detected, determine the pharmacokinetic parameters: maximum concentration, half-life, area under the curve, clearance, volume of distribution.

The FAP20 with BZ371A in a concentration of 5 mg/mL, applied to the genitalia region (whole penis, including glans; and clitoris, majora and minora labia), in volume of 1.5 mL proved to be safe and with good tolerability. There was no clinically significant change regarding safety outcomes related to laboratory tests, vital signs, ECG and physical examination.

Regarding pharmacokinetics, bioanalytical results demonstrated that there were no quantifiable plasma concentrations (Limit inferior of quantification (LIQ)=50 ng/ml) of BZ371A and there were no detectable concentrations (Limit of detection=40 ng/ml) of its fragments in any of the collections performed in the 12 subjects.

In conclusion, FAPP20 was proved to be safe and tolerable in humans, and at the same time avoided the systemic exposure of BZ371A and its fragment.

### REPRESENTATIVE SEQUENCE

The amino acid sequence of peptide PnPP-19 (SEQ ID NO: 1) described by the disclosure is as shown below:
GERRQYFWIAWYKLANSKK

What is claimed is:

1. A topical pharmaceutical formulation comprising:
(i) a synthetic peptide comprising or consisting of the amino acid sequence set forth in SEQ ID NO: 1 present in a concentration of between 0.1% and 0.7% w/v;
(ii) a surfactant;
(iii) propylene glycol; and
(iv) a buffer having a pH between 4.0 and 6.5.

2. A topical pharmaceutical formulation comprising:
(i) a synthetic peptide consisting of the amino acid sequence set forth in SEQ ID NO: 1 present in a concentration between 0.1 and 0.7% w/v;
(ii) a surfactant present in a concentration range of about between 2.5 and 18.0% w/v;
(iii) propylene glycol present in a concentration range of about between 7.5 and 30.0% w/v; and
(iv) a buffer present in a concentration range of about between 50.0 and 73.5% v/v;
wherein the pH of the buffer is of about between 4.0 and 6.5.

3. The topical pharmaceutical formulation according to claim 1 or 2, wherein the surfactant is a poloxamer, preferably wherein the poloxamer comprises Poloxamer 188, Poloxamer 407 or a combination thereof.

4. The topical pharmaceutical formulation according to any one of claims 1 to 3, wherein the buffer comprises sodium acetate or potassium phosphate.

5. The topical pharmaceutical formulation according any one of claims 1 to 4, further comprising a preservative or antioxidant.

6. The topical pharmaceutical formulation according to any one of claims 1 to 5, further comprises one or more additional excipients, optionally wherein the one or more additional excipients comprises a filler, lubricant, wetting agent, flavoring agent, perfume, and/or emulsifier.

7. The topical pharmaceutical formulation according to any one of claims 1 to 6, wherein the formulation is a cream, gel, lotion, ointment, or spray.

8. The topical pharmaceutical formulation according to any one of claims 1 to 7, wherein the formulation crosses the fibrous layer of a subject's tunica albuginea without exhibiting a systemic effect.

9. A method for treating a disease that benefits from modulation of smooth muscle cell contractility, the method comprising topically administering to a subject in need thereof, the formulation according to any one of claims 1 to 8.

10. The method of claim 9, wherein the disease that benefits from modulation of smooth muscle cell contractility is a male sexual dysfunction or a female sexual dysfunction.

11. The method of claim 9, wherein the male sexual dysfunction is selected from the group consisting of erectile dysfunction, delayed ejaculation, premature ejaculation, and male hypoactive sexual desire disorder.

12. The method of claim 9, wherein the female sexual dysfunction is selected from the group consisting of female sexual arousal disorder, female orgasmic disorder, female hypoactive sexual desire disorder and genito-pelvic pain/penetration disorder, optionally wherein the genito-pelvic pain/penetration disorder is dyspareunia or vaginismus.

13. A method for treating male sexual dysfunction, the method comprising topically administering to a subject in need thereof, the formulation according to any one of claims 1 to 7.

14. The method according to claim 13, wherein the male sexual dysfunction is selected from the group consisting of erectile dysfunction, delayed ejaculation, premature ejaculation, and male hypoactive sexual desire disorder.

15. A method for treating female sexual dysfunction, the method comprising topically administering to a subject in need thereof, the formulation according to any one of claims 1 to 7.

16. The method according to claim 15, wherein the female sexual dysfunction is selected from the group consisting of female sexual arousal disorder, female orgasmic disorder, female hypoactive sexual desire disorder and genito-pelvic pain/penetration disorder, optionally wherein the genito-pelvic pain/penetration disorder is dyspareunia or vaginismus.

17. The method according to any one of claims 9 to 16, wherein the administering comprises administration to the subject's skin, mucosa, or urethra.

18. The method of according to claim 13 or 14, wherein the subject is male, and the formulation is administered to the subject's penis or urethra.

19. The method according to claim 15 or 16, wherein the subject is female, and the formulation is administered to the subject's genitalia, optionally wherein the formulation is administered to the subject's labia minora and majora, and/or clitoris.

20. The method according to any one of claims 9 to 19, wherein the administration comprises daily application of the formulation to the subject.

21. A method of making a pharmaceutical formulation according to any one of claims 1 to 8, the method comprising introducing to a pharmaceutically acceptable excipient a peptide consisting of the amino acid sequence set forth in SEQ ID NO: 1, wherein the peptide is present in an amount between about 0.1% and 0.7% w/v.
